(19) United States
(12) Patent Application Publication
(10) Pub. No.: US 2018/0117278 A1
(43) Pub. Date: May 3, 2018

(54) Catheter Apparatus

(71) Applicant: Blaine S. Cameron, Plantation, FL (US)

(72) Inventor: Blaine S. Cameron, Plantation, FL (US)

(21) Appl. No.: 15/338,316

(22) Filed: Oct. 28, 2016

(51) Int. Cl. A61M 25/00

(52) U.S. Cl. CPC A61M 25/0026; A61M 25/007; A61M 25/0017

(57) ABSTRACT

A catheter apparatus formed for insertion into a patient's body cavity and/or subcutaneous tissue, the catheter apparatus has an outer cannula that forms an outer lumen, the outer cannula has a length extending to distal portion, the catheter apparatus includes tubular proximal ends for directing the flow of substances to and from a patient's body.

### FIELD OF THE INVENTION

The present invention relates to a urinary catheter apparatus, and more particularly to a urinary catheter apparatus with anesthetic delivery portion for pain elimination and/or reduction.

### BACKGROUND OF THE INVENTION

Catheter tubing and other invasive tubing are widely implemented in the course of medical treatment. Catheter tubing is generally a hollow, tubular, medical grade, medical equipment inserted into the body to treat diseases or aid in the performance of a surgical procedure. The catheter tubing can be utilized to facilitate the removal or injection of fluids.

Catheter tubing is regularly used for cardiovascular, urological, gastrointestinal, neurovascular, and ophthalmic applications. Yet further, insertion catheters are commonly utilized to direct medical devices into a body cavity, such as a trachea tube.

In many medical applications, the catheter tubing is inserted for extended periods. While local anesthesia is commonly applied prior to the insertion of the catheter tubing about the body cavity, the local anesthesia wears off very quickly resulting in long term patient discomfort or pain. At the time the tubing is subsequently removed from the body, the patient commonly experiences extreme discomfort and pain, at least due to swelling and irritation, as the cavity and nerves are no longer anesthetized. As a result, it is not uncommon in many procedures to unnecessarily apply general anesthetize to render the patient unconscious to avoid feeling such discomfort and pain.

Up until this point, in order for medical professionals to administer local anesthesia to the area contacted by the catheter tubing, the catheter must first be removed and then reinserted. That removal alone is widely done without local anesthesia causing the patient severe discomfort and pain.

Thus, there is a need in the art for a catheter apparatus capable of delivering anesthesia locally to the areas of the body directly or indirectly contacting the catheter tubing.

### SUMMARY OF THE INVENTION

The present invention advantageously provides a catheter apparatus with a portion having the ability to provide local anesthesia or other desired medicine.

According to an embodiment of the present invention, a principal object is to provide a catheter that comprises an outer cannula housing a catheter tube, the outer cannula includes a plurality of medication dispensing portions for excreting mediation from an outer surface of the outer cannula; a drug administration port joined at a proximal end of the outer cannula, the drug administration port is formed in fluid communication with a drug administration channel capable of supplying medication to one or more medication dispensing portions; and a central port joined at the proximal end of the outer cannula, the central port is in fluid communication with a central channel and at least one distal aperture capable of facilitating drainage through the catheter tube.

According to an embodiment of the present invention, another object is to provide a balloon injection portion for facilitating injection and withdrawal of fluid from a balloon positioned towards the at least one distal aperture.

According to an embodiment of the present invention, another object is to provide a balloon injection tube disposed between the outer cannula and the catheter tube, the balloon injection portion further includes a balloon injection port about the proximal end of the outer cannula, the balloon injection tube forming a balloon channel for facilitating the injection and withdrawal of fluid thereof.

According to an embodiment of the present invention, another object is to provide at least a portion of the outer cannula formed contiguously around the central catheter tube. In this manner, the circumference (or any shape) of the tube is contiguous without a break. Another object of one embodiment is to provide a contiguous body in length, a contiguous body in width and a contiguous body in circumference.

According to an embodiment of the present invention, another object is to provide a catheter apparatus comprising at least any two of: an outer cannula; a balloon tube; and a catheter tube; the outer cannula housing capable of housing the balloon tube and the catheter tube, the outer cannula includes a plurality of medication dispensing portions for excreting mediation from an outer surface of the outer cannula; a drug administration port joined at a proximal end of the outer cannula, the drug administration port in fluid communication with a drug administration channel capable of supplying medication to at least one of the plurality of the medication dispensing portions; and a central port joined at the proximal end of the outer cannula, the central port in fluid communication with a central channel and at least one distal aperture capable of facilitating drainage through the catheter tube.

According to an embodiment of the present invention, another object is to provide the outer cannula formed of at least a portion of the drug administration channel.

According to an embodiment of the present invention, another object is to provide at least one contiguous catheter tube having a distal end and a proximal end, the at least one contiguous catheter tube having a dispensing portion disposed about a portion of a length of the at least one contiguous catheter tube, a distal aperture disposed about the distal end, the dispensing portion capable of dispensing medicine via a plurality of delivery apertures, the at least one contiguous catheter tube further including: at least one injection port disposed at the proximal end; and at least one drainage port disposed at the proximal end; the tube further capable of separately transporting medication from the at least one injection port to the dispensing portion, and transporting bodily fluids from the distal aperture to the at least one drainage port.

Other objects will become evident as the present invention is described in detail below.

### BRIEF DESCRIPTION OF THE DRAWINGS

Embodiments of the present application are described herein in which similar elements are given similar reference characters, and a more complete understanding of the present invention, and the attendant advantages and features thereof, will be more readily understood by reference to the following detailed description when considered in conjunction with the accompanying drawings wherein:

FIG. 1 is a perspective view of one embodiment of a catheter apparatus in accordance with the principles of the present invention;

FIG. 2 is a cross-sectional view taken along line A-A of FIG. 1;

FIG. 3 is a cross-sectional view taken along line B-B of FIG. 1;

FIG. 4 is a partial perspective cross-sectional view taken along line B-B of FIG. 1;

FIG. 5 is a perspective view of one embodiment of a catheter apparatus in accordance with the principles of the present invention; and

FIG. 6 is a cross-sectional view taken along line C-C of FIG. 5.

### DETAILED DESCRIPTION OF THE INVENTION

The present invention advantageously provides a catheter apparatus with medicine dispensing portion for dispending medicine about an outer surface of the catheter apparatus. The present invention contemplates various types of catheter apparatus, and as such the term catheter apparatus includes any and all types of invasive tubing, including without limitation intubation tubes, dilation catheters, drainage tubes, or the like.

Accordingly, the apparatus components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

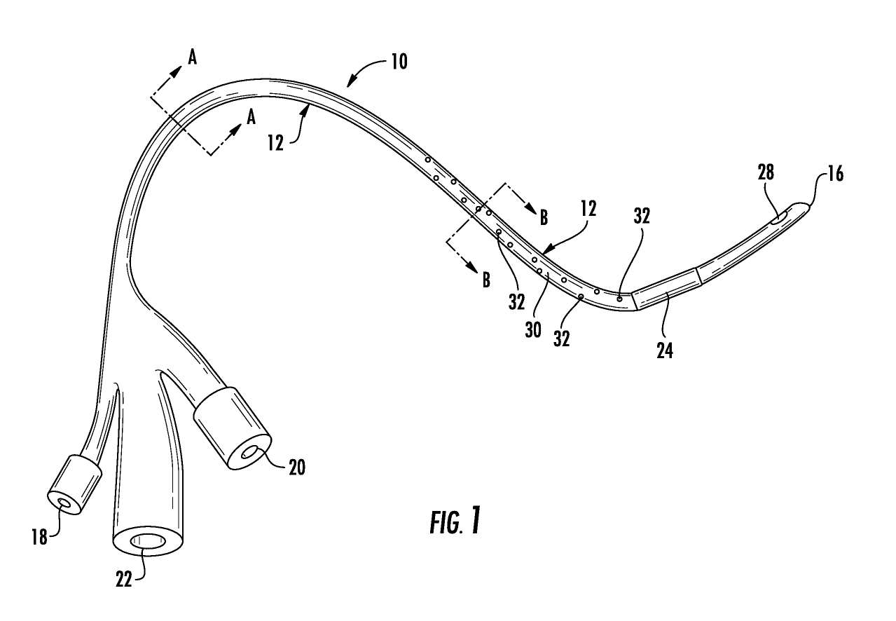

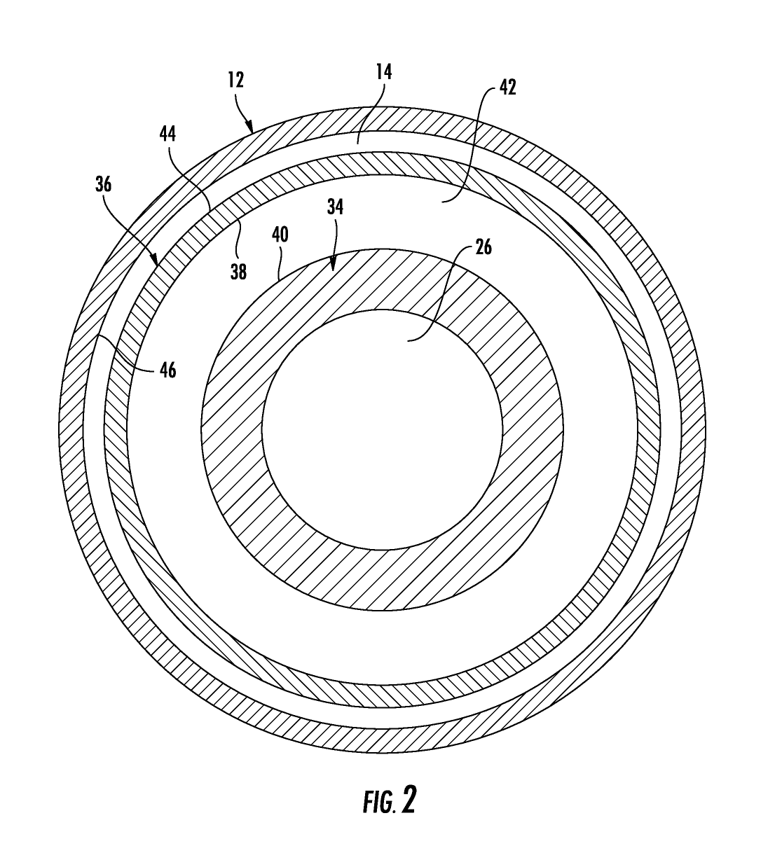

Referring now to the drawings figures in which like reference designators refer to like elements. FIG. 1 shows an exemplary catheter apparatus constructed in accordance with the principles of the present invention and designated generally as catheter apparatus 10. In particular, catheter apparatus 10 is formed for insertion into a patient's body cavity and/or subcutaneous tissue. Subcutaneous tissue means tissue beneath the skin. Catheter apparatus 10 is formed having an outer cannula 12 that forms an outer lumen 14 (FIG. 2), the outer cannula 12 has a length extending to distal portion 16. In the instant embodiment, the catheter apparatus 10 includes three tubular proximal ends defining a balloon injection port 18, a drug administration port 20, and a central port 22.

The balloon injection port 18 is constructed and arranged to accept injection and withdrawal of a liquid for causing the pneumatic and/or hydraulic inflation and deflation of a balloon 24 about the catheter apparatus 10. The balloon 24 is engageable about a patient's body for enlarging or narrowing the opening or passage within the patient's body. The balloon 24 expands outward via injection of liquids and can be retracted inward via withdrawal of the liquids.

Referring to FIG. 2, the central port 22 provides access to a central lumen 26 for facilitating injection and/or drainage of liquids that enter and exit through one or more distal apertures 28. The central lumen 26 is formed at least partially within a central catheter tube 34.

A balloon tube 36 is at least partially disposed between the outer cannula and the central catheter tube 34. The balloon tube 36 has an inner balloon tube surface 38, and the central catheter tube 34 has an outer catheter tube surface 40, the inner balloon tube surface 38 and the outer catheter tube surface 40 forms a middle lumen 42 therebetween for facilitating the passage of substance and/or fluid therethrough. The balloon tube 36 has an outer balloon tube surface 44, and the outer cannula has an inner cannula surface 46, the outer balloon tube surface 44 and the inner cannula surface 46 forms the outer lumen 14.

In one non-limiting alternative embodiment, the central port 22, central lumen 26, and distal aperture 28 facilitates entry and/or maintenance of a central tube (not shown), such as an additional catheter passing or existing therein.

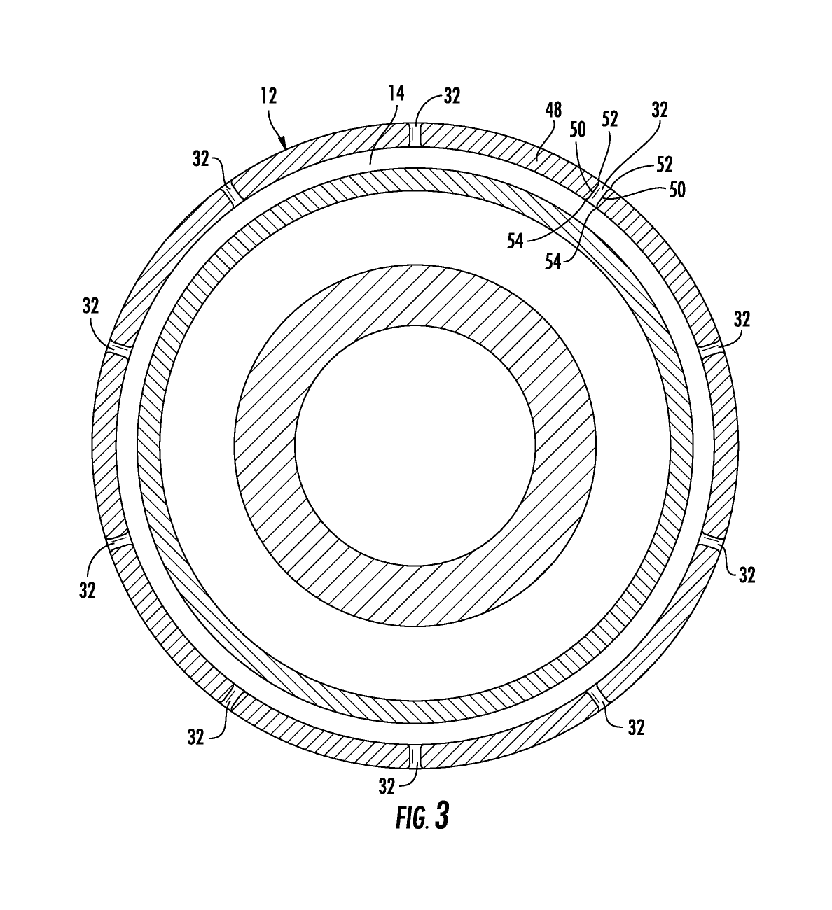

Referring to FIGS. 1 and 3, the drug administration port 20 is constructed and arranged for delivering medicine out from an outer surface 30 of the outer cannula 12. The outer cannula 12 includes a plurality of drug administration apertures 32 that provides a passage way from the outer lumen 14 to the outer surface 30. The outer cannula 12 forms a flexible enclosure 48. The flexible enclosure includes vertical edge walls 50 extending between upper edge walls 52 and lower walls 54.

In one embodiment, the drug administration aperture acts as a one-way valve permitting administration of medicine in one direction from the outer lumen 14 towards the outer surface 30 and otherwise preventing the inward flow of substance and/or fluids, including bodily fluids, from outside towards the outer lumen 14. In this matter, administration of medication, including without limitation anesthesia, will permeate medication or other substance into the area of the body in which the catheter apparatus 10 is disposed. It will be appreciated that within the spirit and scope of the instant invention, additional equivalent structures may be implemented with the outer cannula 12 for providing a one-way valve for dispensing mediation from the catheter apparatus 10.

FIG. 3 further illustrates the central lumen 26 that facilitates passage of fluids between the central port 22 (FIG. 1) and distal aperture 28 (FIG. 1). By way of further example, FIG. 3 illustrates a cross-sectional view of a portion of the central catheter tube 34, the balloon tube 36, and the outer cannula 12.

Referring again to FIG. 1, the outer cannula 12 further includes a balloon 24 that extends via injection of liquids.

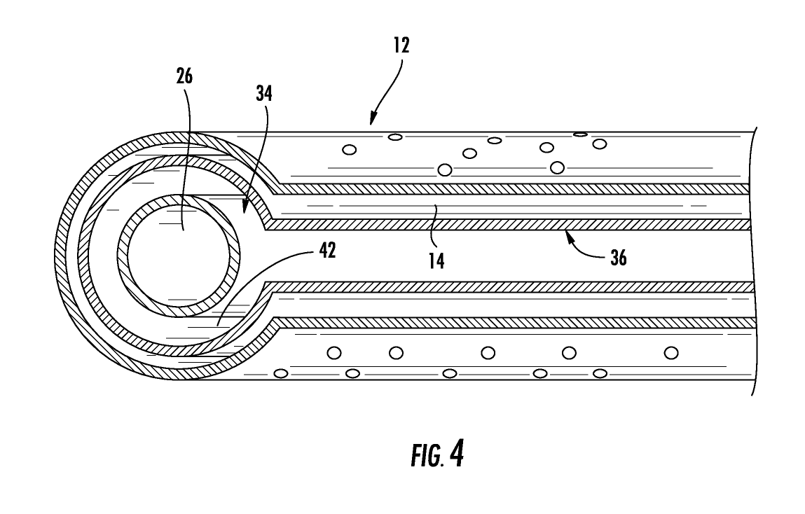

Referring now to FIG. 4, the outer lumen 14 is illustrated between outer cannula 12 and the balloon tube 36, the middle lumen 42 is illustrated between the balloon tube 36 and the central catheter tube 34, and the central lumen 26 is illustrated within the central catheter tube 34. The overall length of the catheter apparatus 10 may vary, including the outer cannula 12, balloon tube 36, and the central catheter tube 34 may vary as desired or required.

In an embodiment, the catheter apparatus 10 including without limitation the outer cannula 12, the balloon tube 36, and the central catheter tube 34, may be formed of any medical grade components and/or suitable materials. For exemplary purposes only, the catheter apparatus may be formed of a polyester interweave, polyethylene, polyurethane, nylon, Teflon, medical grade plastics, medical grade rubbers, medical grade polymer blends, Polyvinvls, fluoropolymers, polyacrylates, polyethersulfons, and like suitable medical grade materials.

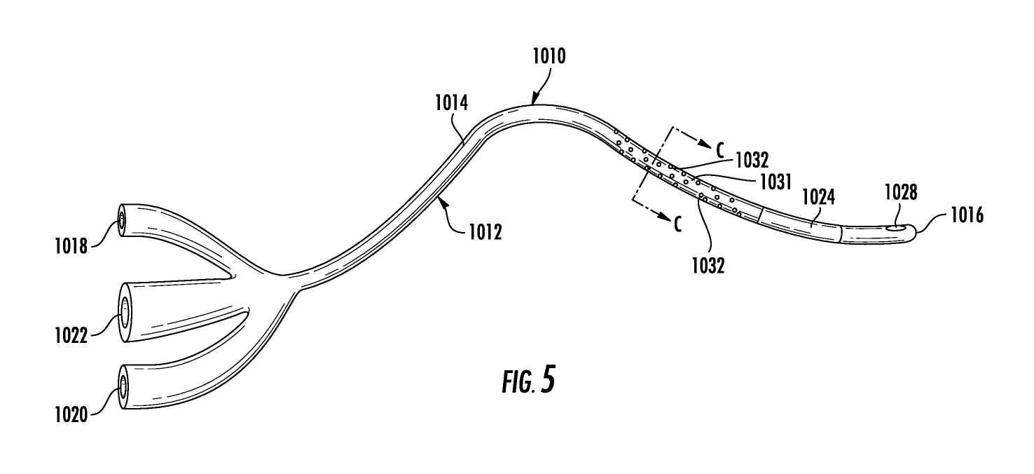

Referring now to FIG. 5, catheter apparatus 1010 is formed for insertion into a patient's body cavity and/or subcutaneous tissue. The catheter apparatus 1010 has a length extending to a distal portion 1016. In one non-limiting embodiment, the catheter apparatus 1010 includes three proximal ends defining a balloon injection port 1018, a drug administration port 1020, and a central port 1022.

The balloon injection port 1018 is constructed and arranged to accept injection and withdrawal of a liquid for causing the pneumatic and/or hydraulic inflation and deflation of a balloon 1024 about the catheter apparatus 1010. The balloon 1024 is engageable about a patient's body for enlarging or narrowing the opening or passage within the patient's body. The balloon 1024 expands outward via injection of liquids and can be retracted inward via withdrawal of the liquids.

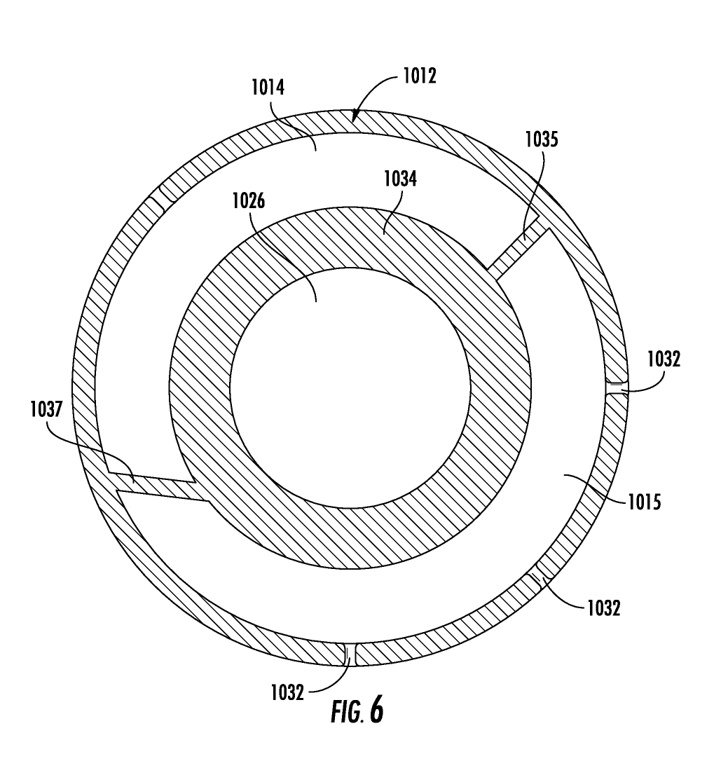

Referring to FIG. 6, catheter apparatus 1010 is formed having an outer cannula 1012 with a first outer lumen 1014 and a second outer lumen 1015.

Referring to FIGS. 5 and 6, the outer lumen 1014 includes a drug dispensing portion 1031. The drug dispensing portion includes a plurality of drug administration apertures 1032 that are in fluid communication with both the drug administration port 1020 and the second outer lumen 1015.

FIG. 6 illustrates the outer lumen 1012 coupled with a central catheter tube 1034. In one embodiment, the outer lumen 1012 couples with the central catheter tube 1034 via one or more walls 1035, 1037. The combination of the outer lumen 1012 with the central catheter tube 1034 and at least two walls 1035, 1037 forms the first and second outer lumen 1014, 1015. In one non-limiting embodiment, the walls are sloped to facilitate the formation of a spiral structure of the first and second lumen 1014, 1015 wrapping around and along at least a portion of the length of the central catheter tube 1034 towards the distal portion 1016. The first and second outer lumen 1014, 1015 provides for separate chambers extending about at least a portion of the length of the catheter apparatus 1010. While the instant embodiment includes a spiral formation of the first and second outer lumen 1014, 1015, it is not so required, and alternate chamber patterns and/or formations are within the spirit and scope of the instant invention.

The first outer lumen 1014 facilitates passage of substance and/or fluid therethrough from which the balloon 1024 expands outward and/or retracts inward by injecting or withdrawing a substance or liquid about the balloon injection port 1018.

The second outer lumen 1015 facilitates the flow of medicine injected about the drug administration port 1020, the plurality of drug administration apertures 1032 are formed to secrete medicine in any form, including without limitation liquid, gel, powder, local, or topical anesthesia.

The central lumen 1026 facilitates passage of fluids between the central port 1022 (FIG. 5) and a distal aperture 1028 (FIG. 5). In a non-limiting embodiment, an additional tube (not shown) may be fed through the central port 1022 out from the distal aperture 1028.

While at least one embodiment herein describes a Foley catheter due to the balloon portion, it should be appreciated that the instant invention described herein can be modified to include any type of catheter and that the balloon portion is not necessary to practice each embodiment described in the instant invention, it will be appreciated by those skilled in the art that the instant invention encompasses alternate catheters as further described herein.

As used herein, when the term “and/or” is used, it shall include all combinations of one or more of the associated described items.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are contemplated in light of the above teachings without departing from the scope and spirit of the invention. It will be readily apparent that various changes may be made thereto without departing from the spirit and scope of the disclosure or sacrificing all of its material advantages. The examples and embodiments described herein are merely exemplary of the instant disclosure.

What is claimed is:

1. A catheter comprising:
an outer cannula housing a catheter tube, said outer cannula includes a plurality of medication dispensing portions for excreting mediation from an outer surface of said outer cannula;
a drug administration port joined at a proximal end of said outer cannula, said drug administration port in fluid communication with a drug administration channel capable of supplying medication to at least one of said plurality of said medication dispensing portions; and
a central port joined at said proximal end of said outer cannula, said central port in fluid communication with a central channel and at least one distal aperture capable of facilitating drainage through said catheter tube.

2. The catheter of claim 1 further comprising a balloon injection portion for facilitating injection and withdrawal of fluid from a balloon positioned towards said at least one distal aperture.

3. The catheter of claim 2 wherein said balloon injection portion includes a balloon injection tube disposed between said outer cannula and said catheter tube, said balloon injection portion further includes a balloon injection port about said proximal end of said outer cannula, said balloon injection tube forming a balloon channel for facilitating the injection and withdrawal of fluid thereof.

4. The catheter of claim 1 wherein at least a portion of said outer cannula is formed contiguously around said central catheter tube.

5. A catheter comprising:
a catheter apparatus comprising at least any two of:
an outer cannula;
a balloon tube; and
a catheter tube;
said outer cannula housing capable of housing said balloon tube and said catheter tube, said outer cannula includes a plurality of medication dispensing portions for excreting mediation from an outer surface of said outer cannula;
a drug administration port joined at a proximal end of said outer cannula, said drug administration port in fluid communication with a drug administration channel capable of supplying medication to at least one of said plurality of said medication dispensing portions; and
a central port joined at said proximal end of said outer cannula, said central port in fluid communication with a central channel and at least one distal aperture capable of facilitating drainage through said catheter tube.

6. The catheter of claim 5 wherein said outer cannula forms at least a portion of said drug administration channel.

7. An indwelling Foley catheter comprising:
at least one contiguous catheter tube having a distal end and a proximal end, said at least one contiguous catheter tube having a dispensing portion disposed about a portion of a length of said at least one contiguous catheter tube, a distal aperture disposed about said distal end, said dispensing portion capable of dispensing medicine via a plurality of delivery apertures, said at least one contiguous catheter tube further including:
at least one injection port disposed at said proximal end; and
at least one drainage port disposed at said proximal end;
said tube further capable of separately transporting medication from said at least one injection port to said dispensing portion, and transporting bodily fluids from said distal aperture to said at least one drainage port.
